(19) United States
(12) Patent Application Publication
(10) Pub. No.: US 2011/0024308 A1
(43) Pub. Date: Feb. 3, 2011

(54) IMMUNOASSAY

(75) Inventor: Masaaki Kobayashi, Kawasaki-shi (JP)

Correspondence Address: FITZPATRICK CELLA HARPER & SCINTO, 1290 Avenue of the Americas, NEW YORK, NY, 10104-3800, US

(73) Assignee: CANON KABUSHIKI KAISHA, Tokyo (JP)

(21) Appl. No.: 12/935,269

(22) PCT Filed: Apr. 15, 2009
(86) PCT No.: PCT/JP2009/057942

§ 371 Date: Sep. 28, 2010

(30) Foreign Application Priority Data
Apr. 17, 2008 (JP) 2008-108155

(51) Int. Cl. G01N 33/543; G01N 27/26; G01N 33/58

(52) U.S. Cl. 205/782; 205/792

(57) ABSTRACT

Provided is an immunoassay for detecting a test substance with high sensitivity. The immunoassay is a method of detecting or measuring a test substance contained in a sample liquid, and includes: reacting, with the test substance, a first specifically binding substance which is immobilized on a support and is capable of binding specifically to the test substance and a second specifically binding substance which is labeled with a labeling substance and is capable of binding specifically to the test substance; separating, from the support, the second specifically binding substance which is unreactive with the test substance; eluting the labeling substance retained on the support; depositing the eluted labeling substance on a test element; and measuring electrochemically a catalyst reaction amount of the labeling substance deposited on the test element.

### TECHNICAL FIELD

The present invention relates to an immunoassay for detecting a test substance.

### BACKGROUND ART

In an immunological measurement using an antigen-antibody reaction, there has widely been used a metal colloid. For example, there are known an immunochromatography method and an agglutination colorimetric method. As an example of the principle of the immunochromatography method, the following is known. An antibody labeled with a metal colloid is reacted with an antigen, whereby an antigen-antibody-metal colloid complex is obtained. The complex is flowed on a determination paper (film) on which an antibody is immobilized. The complex is captured by the immobilized antibody when the complex is present, and as a result, coloration due to the metal colloid is generated at a predetermined position on the determination paper. By determining the coloration, the presence or absence of the antigen can be recognized. On the other hand, as an example of the principle of the agglutination colorimetric method, the following is known. When an antibody bound with a gold colloid is reacted with an antigen in a solution, a color tone is changed as a result of agglutination of the gold colloid. Therefore, the presence or absence of the antigen or the amount thereof can be determined by measuring absorbance. However, those immunological measurement methods each using a metal colloid have not necessarily satisfied a sensitivity required for an assessment.

As a method for solving the problem, there is proposed, in Analytical Chemistry, 2000, 72, 5521 to 5528 (Non-patent Document 1), a method of electrochemically measuring a gold colloid. In this method, first, a primary antibody immobilized on a support and a secondary antibody labeled with a gold colloid are reacted with an antigen, whereby a primary antibody-antigen-secondary antibody complex is formed. Then, the gold colloid is dissolved by an oxidation reaction to be changed into chloroauric acid (gold ion), and the resultant is further deposited on a test element by electrodeposition. After that, an electrochemical oxidation reaction of gold deposited on the test element is measured, to thereby assay the concentration of the antigen.

Further, in order to achieve higher sensitivity in the electrochemical gold colloid immunoassay such as the one described in Non-patent Document 1, there is proposed, in Analytica Chemica Acta 538 (2005) 159 to 164 (Non-patent Document 2), a method of amplifying a signal amount by subjecting the gold colloid to crystal growth. In this method, first, a primary antibody immobilized on a support and a secondary antibody labeled with a gold colloid are reacted with an antigen, whereby a primary antibody-antigen-secondary antibody complex is formed. Next, a gold ion solution is added thereto, and the gold colloid contained in the complex is subjected to crystal growth. After that, in the same manner as in the above Non-patent Document 1, an electrochemical oxidation reaction of gold is measured, to thereby assay the concentration of the antigen.

### DISCLOSURE OF THE INVENTION

The immunological measurement method described in Non-patent Document 1 uses the electrochemical oxidation reaction of gold as an index, and thus, the detection sensitivity of a test substance is more enhanced when a gold colloid having a larger particle diameter is used as a labeling substance. The fact also applies to, in the process of electrodepositing the gold ion in the method of Non-patent Document 1, the case of detecting an antigen by electrochemically measuring a reduction reaction of the gold ion.

However, on the other hand, the dispersibility and the stability of the secondary antibody labeled with a gold colloid become poorer as the particle diameter of the gold colloid becomes larger. Therefore, the particle diameter of the metal colloid to be used in the immunological measurement is generally 5 to 80 nm.

In Non-patent Document 1, a gold colloid having a particle diameter of about 18 nm is used as the labeling substance, and there remains a problem in the detection sensitivity.

On the other hand, in the method described in Non-patent Document 2, the signal amplification is performed by subjecting the gold colloid to crystal growth after an immune reaction. A test substance can be certainly detected with high sensitivity by the method, but it takes 90 minutes for the growth reaction of the gold colloid in the gold ion solution, which is a significant problem from the viewpoint of rapidity.

The present invention has been made in view of solving the above problems, and the present invention provides a method of detecting or measuring a test substance contained in a sample liquid.

That is, the present invention is an immunoassay for detecting or measuring a test substance contained in a sample liquid, including: reacting, with the test substance, a first specifically binding substance which is immobilized on a support and is capable of binding specifically to the test substance and a second specifically binding substance which is labeled with a labeling substance and is capable of binding specifically to the test substance; separating, from the support, the second specifically binding substance which is unreactive with the test substance; eluting the labeling substance retained on the support; depositing the eluted labeling substance on a test element; and measuring electrochemically a catalyst reaction amount of the labeling substance deposited on the test element, in which the labeling substance has an action of catalyzing an electrochemical reaction of a solution having the test element as an electrode.

According to the immunoassay of the present invention, a test substance can be detected quickly with high sensitivity only by measuring electrochemically the catalyst reaction amount of the labeling substance.

### BRIEF DESCRIPTION OF THE DRAWINGS

FIGS. 1A, 1B, 1C, and 1D are conceptual diagrams illustrating a determination method of a test substance using a particle as a support according to an embodiment of the present invention.

FIGS. 2A, 2B, and 2C are conceptual diagrams illustrating a determination method of a test substance using a test element as a support according to an embodiment of the present invention.

FIG. 3 is a graph illustrating a result obtained by electrochemically measuring a reduction current of gold to be deposited on an electrode according to a comparative example of the present invention.

FIG. 4 is a graph illustrating a result obtained by electrochemically measuring a hydrogen overvoltage of gold deposited on an electrode according to an example of the present invention.

FIG. 5 is a graph illustrating a result obtained by comparing a catalytic capacity of each of labeling substances according to an embodiment of the present invention.

FIG. 6 is a graph illustrating a detection result of HCG when a particle is used as a support according to an embodiment of the present invention.

FIG. 7 is a graph illustrating a detection result of HCG when a test element is used as a support according to an embodiment of the present invention.

### BEST MODE FOR CARRYING OUT THE INVENTION

The present invention provides an immunoassay for detecting or measuring a test substance contained in a sample liquid, including: reacting, with the test substance, a first specifically binding substance which is immobilized on a support and is capable of binding specifically to the test substance and a second specifically binding substance which is labeled with a labeling substance and is capable of binding specifically to the test substance; separating, from the support, the second specifically binding substance which is unreactive with the test substance; eluting the labeling substance retained on the support; depositing the eluted labeling substance on a test element; and measuring electrochemically a catalyst reaction amount of the labeling substance deposited on the test element. As the labeling substance, there is used a substance having an action of catalyzing an electrochemical reaction of a solution having the test element as an electrode.

Those processes are generally performed in a solution. In general, the processes are performed in the following procedure: the labeling substance contained in a complex composed of the first specifically binding substance, the test substance, and the second specifically binding substance is deposited on the test element by the elution/deposition reaction; and the catalyst reaction amount of the deposited labeling substance is detected by the electrochemical measurement method to be described below. That is, when the amount of the test substance contained in the sample liquid is larger, the amount of the labeling substance deposited on the test element becomes larger, and hence, the catalyst reaction amount of the labeling substance also increases and the amount of the test substance can be measured.

It is preferred that the measurement of the catalyst reaction amount be performed in an aqueous solution and that the labeling substance catalyze the electrolysis reaction of water. Further, it is preferred that the labeling substance be a conductive material having a potential window narrower than a potential window of the test element.

An outline of an embodiment of a measurement method according to the present invention is described by using diagrams. It should be noted that, in this embodiment, the detection can be performed in accordance with the following procedure, but the embodiment is not limited thereto.

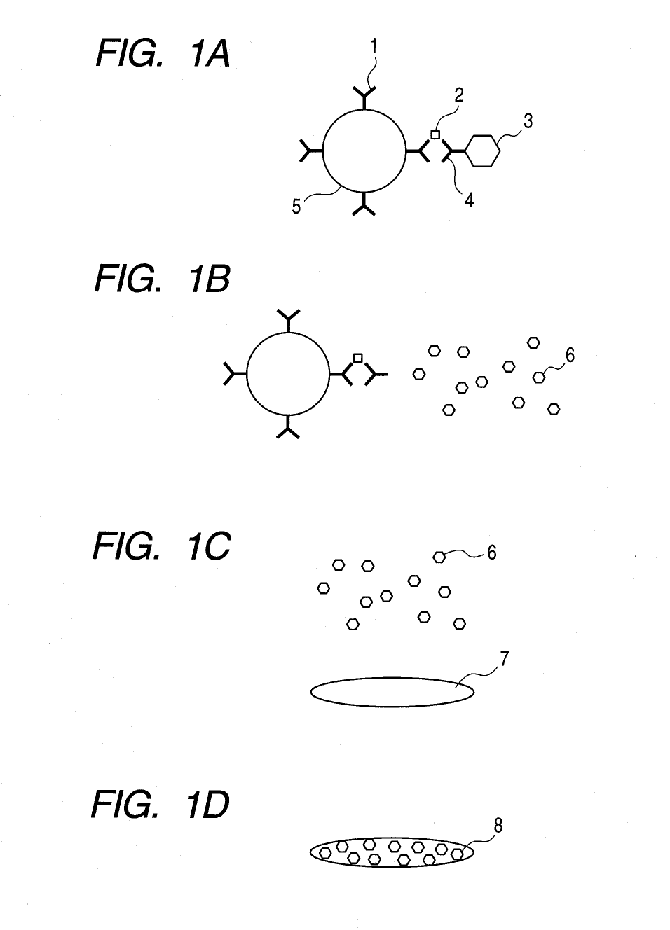

FIGS. 1A, 1B, 1C, and 1D illustrate a method of detecting or measuring a test substance in a sample by using a carrier particle as a support 5.

First, a sample liquid and a first specifically binding substance 1 immobilized on the support 5 are mixed to thereby form a complex composed of a test substance 2 and the first specifically binding substance 1 immobilized on the support 5. Next, an unreacted product in the sample liquid is removed by a washing operation such as B/F separation to thereby form a complex of the complex composed of the test substance 2 and the first specifically binding substance 1 immobilized on the support 5 and a second specifically binding substance 4 labeled with a labeling substance 3. Then, an unreacted second specifically binding substance 4 labeled with the labeling substance 3 is removed. By the series of those processes, a complex composed of the first specifically binding substance, the test substance, and the second specifically binding substance is formed. FIG. 1A illustrates the complex.

It should be noted that, in the process of forming the complex illustrated in FIG. 1A, there may be used a process of simultaneously or almost simultaneously performing the mixing of the sample liquid and the first specifically binding substance 1 immobilized on the support 5 and the mixing of the sample liquid and the second specifically binding substance 4 labeled with the labeling substance 3.

Next, to the complex illustrated in FIG. 1A, an effluent for eluting the labeling substance in the complex is added, to thereby elute the labeling substance. After that, a solution of an eluted labeling substance 6 and a test element are brought into contact with each other. FIG. 1B illustrates elution of the labeling substance 3 in the complex, and FIG. 1C illustrates a state where the test element and the eluted labeling substance 6 are brought into contact with each other. Then, as illustrated in FIG. 1D, the eluted labeling substance 6 is deposited on a test element 7 by electrodeposition, thereby electrochemically measuring a catalyst reaction amount of a reaction which is catalyzed by a labeling substance 8.

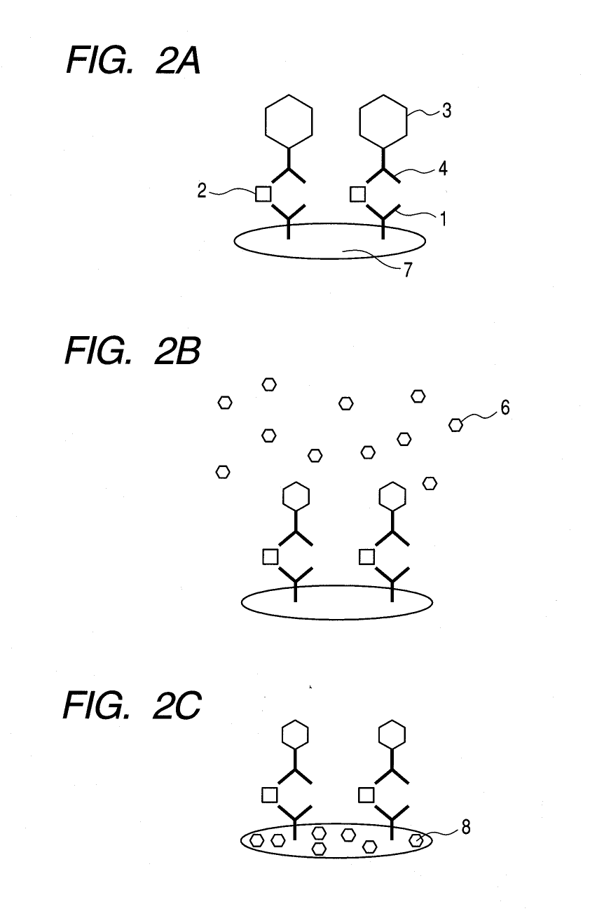

FIGS. 2A, 2B, and 2C illustrate a method of detecting or measuring a test substance in a sample by using a test element 7 as a support.

First, a sample liquid and a first specifically binding substance 1 immobilized on the test element 7 are brought into contact with each other to thereby form a complex composed of a test substance 2 and the first specifically binding substance 1 immobilized on the test element 7.

Next, an unreacted product in the sample liquid is removed by a washing operation such as B/F separation to thereby form a complex of the complex composed of the test substance 2 and the first specifically binding substance 1 immobilized on the test element 7 and a second specifically binding substance 4 labeled with a labeling substance 3. Then, an unreacted second specifically binding substance 4 labeled with the labeling substance 3 is removed. By the series of those processes, there is formed a complex composed of the first specifically binding substance, the test substance, and the second specifically binding substance. FIG. 2A illustrates the complex.

It should be noted that, in the process of forming the complex illustrated in FIG. 2A, there may be used a process of simultaneously or almost simultaneously performing the mixing of the sample liquid and the first specifically binding substance 1 immobilized on the test element 7 and the mixing of the sample liquid and the second specifically binding substance 4 labeled with the labeling substance 3.

Next, electroelution is performed using the test element 7 to thereby elute the labeling substance in the complex illustrated in FIG. 2A. FIG. 2B illustrates the elution of the labeling substance 3 in the complex. Next, as illustrated in FIG. 2C, electrodeposition is performed using the test element 7, to thereby deposit an eluted labeling substance 6 on the test element 7. Then, a catalyst reaction amount of a reaction which is catalyzed by a deposited labeling substance 8 is electrochemically measured.

Next, an electrochemical measurement of the present invention is described.

In an electrochemical system (combination of solvent, supporting electrolyte, and electrode), the oxidation of water is proceeded at a sufficiently positive potential, which generates oxygen and allows a large Faradaic current to flow. The magnitude of the deviation of the electrode potential at which oxygen is actually generated from the thermodynamic value is referred to as oxygen overvoltage. On the other hand, the reduction of water is proceeded at a sufficiently negative potential, which generates hydrogen and allows a large Faradaic current to flow. The magnitude of the deviation of the electrode potential at which hydrogen is generated from the thermodynamic value is referred to as hydrogen overvoltage. The oxygen overvoltage and the hydrogen overvoltage each differ depending on the kind of electrode materials. When the catalytic capacity at the time of the electrolysis reaction of water, which a labeling substance deposited on an electrode has, is higher, the oxygen overvoltage and the hydrogen overvoltage become smaller. A potential window means a potential region which is not influenced by those oxygen and hydrogen overvoltages. Further, the width of the potential window generally depends on the physical properties of an electrode to be used and the surface condition of the electrode. For example, in the case of a comparison in terms of the hydrogen overvoltage, the potential window of a diamond thin film electrode or a carbon electrode is wide, and the potential window of gold or platinum is narrow when compared thereto. In other words, the catalytic capacity of a hydrogen ion is low in a diamond thin film and carbon, and the catalytic capacity of the hydrogen ion is high in gold and platinum. Thus, when a labeling substance such as gold or platinum is deposited on an electrode made of the diamond thin film or carbon, the potential window of the electrode in such a state becomes, in accordance with the deposition amount, narrower than that of the electrode before the deposition. Therefore, a test substance can be detected by measuring the difference between the potential windows before and after the deposition of the labeling substance. In the case where water is used as a solvent, a potential window refers to a potential region where the generation of oxygen due to the oxidation of water does not occur at a positive potential side and to a potential region where the generation of hydrogen due to the reduction of water does not occur at a negative potential side. The measurement of the difference between the potential windows may be performed by measuring at least one of the potential window of the positive potential side and the potential window of the negative potential side. In the case where a cathode electrode is used as a test element, the change in the hydrogen overvoltages before and after the deposition of the labeling substance may be measured, and in the case where an anode electrode is used as a test element, the change in the oxygen overvoltages before and after the deposition of the labeling substance may be measured.

Here, there is considered the change in the hydrogen overvoltage which occurs at the time when gold is deposited on a carbon electrode. For example, the hydrogen overvoltage of a carbon electrode a part on which gold is deposited becomes smaller than the hydrogen overvoltage of a carbon (on which gold is not deposited at all) electrode alone. Further, as the amount of gold deposited on carbon is increased, the hydrogen overvoltage becomes further smaller. A value of the hydrogen overvoltage is an applied potential which is larger than a potential obtained from a thermodynamic calculation. An actual value of the hydrogen overvoltage can be determined from, after a current-potential curve is measured, a potential at which a reduction current of hydrogen generation begins to flow or a potential at which an air bubble of hydrogen begins to be generated on the electrode.

Further, the catalytic capacity of a labeling substance can be measured as a change in the hydrogen overvoltage or the oxygen overvoltage of an electrode, which is brought about by the deposition of the labeling substance, and at the same time, because the deposition of the labeling substance causes a change in the reaction rate of a reaction which the labeling substance catalyzes, the catalytic capacity can also be determined by electrochemically measuring the reaction rate. That is, as shown in examples, a current-potential curve is measured, and a change in the amount of the current at a constant voltage value can be measured. The constant voltage value in this case is set in a potential region which is at outer side of the potential window of water as the material of the test element. Therefore, the constant voltage value is selected from a potential region lower than the hydrogen overvoltage of the test element in the case of the negative potential side and a potential region higher than the oxygen overvoltage of the test element in the case of the positive potential side, and the change in the amount of the current at the constant voltage value is measured.

Therefore, the measurement of the change in the hydrogen overvoltage includes not only determining directly the value of the hydrogen overvoltage, but also the case where the current at a constant voltage value is measured.

The change in the hydrogen overvoltage is a change caused by the catalytic capacity of gold described above. The present invention has taken the phenomenon into account, and has attempted to detect the deposition amount of a substance deposited on an electrode as a change in the hydrogen overvoltage. Then, as a result of intensive studies, it was found that deposited gold can be detected with higher sensitivity by measuring the hydrogen overvoltage that gold itself has, rather than by measuring the electrochemical oxidation/reduction reaction of gold itself.

That is, it means that a test substance can be detected with higher sensitivity by electrochemically measuring a catalyst reaction which deposited gold catalyzes, rather than by electrochemically measuring the oxidation/reduction reaction of gold deposited on the electrode as described in Non-patent Document 1.

In addition, the present invention can pick up an electrochemical signal from hydrogen ions large amount of which are contained in a measurement solution, by utilizing the catalyst reaction of gold, and thus is excellent in signal amplification ability. Therefore, the growth reaction of the gold colloid as described in Non-patent Document 2 becomes unnecessary.

The catalyst reaction amount of a labeling substance according to the present invention refers to the amount of an electrochemical reaction which is a target catalyzed by the labeling substance, and which proceeds between an electrode and a chemical substance in a solution, and refers preferably to the amount of an electrolysis reaction of water in the case of using an aqueous solution. The catalyst reaction amount can be expressed as the current value indicating the reaction rate of the reaction or as the magnitude of the electrochemical oxygen and hydrogen overvoltages caused at the time when a labeling substance is deposited on a test element. Further, the process of electrochemically measuring the difference between the potential windows may be a process of electrochemically measuring the difference between the oxygen and hydrogen overvoltage values of the test element and the oxygen and hydrogen overvoltage values of the test element on which the labeling substance is deposited.

Here, in the description above, there were used the carbon electrode as the test element and gold as the labeling substance, but the present invention is not limited thereto. Any combination may be used in the present invention as long as there is a difference in the potential window between the test element and the test element on which the labeling substance is deposited, and there may be considered various combinations such as a carbon electrode and platinum, and a diamond thin film electrode and gold.

As an electrochemical measurement method, any method can be employed as long as it can measure at least one of the oxygen overvoltage value and the hydrogen overvoltage value, and examples thereof include a constant-potential measurement method, a constant-current measurement method, linear sweep voltammetry (LSV), differencial pulse voltammetry (DPV), square wave voltammetry (SWV), chrono amperometry (CA), chrono coulometry (CC), and cyclic voltammetry (CV).

As the applications of the present invention, there are exemplified a urine analysis, a pregnancy test, a blood test, a water quality test, a stool test, a soil analysis, and a food analysis.

The sample to which the detection method of the present invention can be applied is not particularly limited as long as the sample has a possibility of containing a test substance, and for example, a biological sample or an environment-derived sample can be given. Examples of the biological sample include body fluids (e.g., blood, blood serum, blood plasma, spinal fluid, sweat, saliva, and urine), hair, excretions, organs, and tissues of animals, animals and plants themselves, or dried products thereof. Examples of the environment-derived sample include river water, lake water, sea water, and soil.

As the test substance and the specifically binding substance of the present invention, an antigen and an antibody can be exemplified. Examples of the antigen include various substances such as a nucleic acid, a protein, a peptide, an amino acid, a saccharide, a cell, an antibody, an antigen, an enzyme, a receptor, and an environmental hormone, and may be a substance known in the field of immunological measurement or a novel substance. Examples of the antibody include an anti-cell antibody, an anti-protein antibody, an anti-glycoprotein antibody, an anti-enzyme antibody, an anti-polysaccharide antibody, an anti-bacterial antibody, and an anti-viral antibody. Further, the antibody may be a monoclonal antibody or a polyclonal antibody, and in addition, the antibody may express an intact molecule, and fragments and derivatives thereof, and may include antibody fragments such as F(ab′)2, Fab′, and Fab. Further, IgG is generally used as the antibody, but there may also be used F(ab′)2, Fab′, and Fab which are obtained by low-molecularizing IgG using a digestive enzyme such as pepsin or papain or a reducing agent such as dithiothreitol or mercaptoethanol. Still further, there may be used not only IgG but also IgM or a fragment obtained by low-molecularizing IgM with the same treatment as that for IgG. Two or more kinds of monoclonal antibodies with different recognition epitopes may also be used in combination. Here, any of the following cases may be adopted: an antigen is used as the test substance and an antibody which binds to the antigen is used as the specifically binding substance; and an antigen is used as the specifically binding substance and an antibody which binds to the antigen is used as the test substance. Further, a first specifically binding substance refers to the specifically binding substance immobilized on a support, and a second specifically binding substance refers to the substance labeled with a labeling substance. That is, the descriptions of “first” and “second” in the specifically binding substances are used in order to distinguish the substance immobilized on the support from the substance labeled with the labeling substance, and do not mean that the substances themselves are different from each other. The first and second specifically binding substances are not particularly limited as long as they coexist with the test substance and do not inhibit each other from binding to the test substance.

The support is a solid phase for capturing and retaining a test substance contained in a sample liquid, separating the test substance as a complex to which a labeling substance is bound from the sample liquid, and performing purification. The support is not particularly limited as long as it is substantially insoluble in solvents such as a sample liquid and a measurement solution and can immobilize the first specifically binding substance thereon. Further, as illustrated in FIGS. 1A, 1B, 1C, and 1D and FIGS. 2A, 2B, and 2C, it is preferred that an insoluble magnetic carrier particle or a test element itself for performing an electrochemical measurement be used as the support when the removal of an unreacted substance by B/F separation or the simplification of the process of testing a test substance is taken into consideration.

A typical insoluble magnetic carrier particle is a fine particle composed of a film phase formed of an organic polymer substance and a core phase formed of a magnetic substance. Examples of the insoluble magnetic carrier particle include latex, gelatin, and liposome, which each contain therein a fine particle formed of a metal such as iron (III) oxide (Fe₃O₄), iron (II) oxide (γ-Fe₂O₃), various ferrites, iron, manganese, nickel, cobalt, or chromium, an alloy formed of cobalt, nickel, or manganese, or a magnetic particle thereof. Favorably exemplified is a latex particle composed of the magnetic substance as a nucleus and a latex film surrounding the nucleus. The latex originally means a milky liquid which exudates from a rubber tree when a cut is made thereto, but the latex used in the present invention means a suspension or an emulsion in which discrete fine particles are suspended in an aqueous liquid. As the insoluble magnetic carrier particle used in the present invention, a fine particle in which the surface of the magnetic particle as a nucleus is subjected to surface treatment with an organic substance is preferable used, but the insoluble magnetic carrier particle used in the present invention is not limited thereto.

Typical insoluble magnetic carrier particles, which are commercially available, include: Dynabeads M-270 Epoxy, Dynabeads M-270 Amine, Dynabeads M-270 Carboxylic Acid, Dynabeads M-270 Tosylactivated, Dynabeads M-450 Epoxy, and Dynabeads M-450 Tosylactivated supplied by VERITAS Corporation; IMMUTEX-MAG supplied by JSR Corporation; and SMG-11 supplied by Fujikura Kasei Co., Ltd., and other insoluble magnetic carrier particles are available from Bangs Laboratories, Inc., and the like.

The insoluble magnetic carrier particle having a particle diameter of 0.01 μm to 20 μm is used, and the insoluble magnetic carrier particle having a particle diameter in the range of 0.1 μm to 6 μm is preferred. As a method of adsorbing or binding a specifically binding substance to the insoluble magnetic carrier particle, there is employed a method of physically adsorbing or binding the specifically binding substance or a method of chemically binding the specifically binding substance.

The labeling substance is not particularly limited as long as the labeling substance deposited on a test element has the catalytic capacity described above and is a substance which can electrochemically measure the amount of the catalytic action thereof. The labeling substance is preferably a conductive material, and the narrower the potential window of the conductive material is, compared with the potential window of the test element, the easier the detection of the change in the potential window of an electrode caused by the deposition of the labeling substance becomes, which is preferred in view of the measurement. Examples thereof may include various metal colloids such as a gold colloid, platinum colloid, silver colloid, palladium colloid, copper colloid, nickel colloid, and indium colloid, and various semiconductor nanoparticles such as CdS, PdS, CuS, and ZnS. The gold colloid and platinum colloid are preferred.

Further, the particle diameter of the labeling substance according to the present invention is not particularly limited as long as the catalyst reaction amount which the labeling substance has can be electrochemically measured. However, in a practical sense, there occur decrease in immunoreactivity and enhancement in agglutinating property depending on the size of the particle diameters at the time of labeling the second specifically binding substance with the labeling substance. Therefore, the particle diameter of the labeling substance is more preferably between 5 nm and 200 nm. As a method of labeling a specifically binding substance with the labeling substance, there is employed a method of physically adsorbing or binding the specifically binding substance or a method of chemically binding the specifically binding substance.

The process of separating an unreacted substance according to the present invention means a washing process (B/F separation) which is widely used in an immunological measurement method typified by an enzyme immunoassay.

For example, in the case where an insoluble magnetic carrier particle to which a primary antibody is bound is used, a sample liquid containing an antigen is added thereto and the reaction with the insoluble magnetic carrier particle is performed for a certain period of time, whereby an antigen-antibody complex is formed. The antigen in this form is referred to as a bound antigen (bound form: B). On the other hand, an antigen which is unreactive with the antibody immobilized on the insoluble magnetic carrier particle is referred to as a free antigen (free form: F). The B/F separation means a process of separating the bound antigen and the free antigen by washing.

Further, the same applies to the case where a secondary antibody labeled with a labeling substance such as a gold colloid is reacted with the bound antigen. A complex composed of the primary antibody-immobilized insoluble magnetic carrier particle, the antigen, and the gold colloid-labeled secondary antibody is referred to as a bound form, an unreacted gold colloid-labeled secondary antibody is referred to as a free form, and the case is handled as B/F separation.

The process of eluting the labeling substance according to the present invention means a process of eluting the labeling substance by adding a solution (effluent) for dissolving the labeling substance. At that time, a voltage may be applied thereto for promoting the elution (electroelution). Further, examples of the effluent include an acid solution of aqua regia, diluted hydrochloric acid, or diluted sulfuric acid, an aqueous solution containing chloride ions, and various etchants.

On the other hand, the process of depositing the labeling substance according to the present invention means a process of depositing the eluted labeling substance on a test element. As a deposition method, there are exemplified various electroless plating methods and electrodeposition methods, and preferred is a electrodeposition method which uses a test element in terms of effectively collecting the eluted labeling substance on the test element.

Here, the elution process and the deposition process of the labeling substance according to the present invention are performed continuously, and thus need caution. For example, in the case where aqua regia is used as the effluent, the labeling substance can be eluted in a short period of time, but in the process of electrodeposition, there occurs a problem that the deposition amount of the labeling substance decreases. Therefore, it is desirable to select a liquid which can effectively cause both dissolving with acid and deposition by a voltage application, and in the case of aqua regia, it may be diluted to 5 to 30%. Further, there is also a method of performing the electrodeposition after the following processes: the labeling substance is dissolved in aqua regia and then an alkaline solution such as sodium hydroxide is added thereto to thereby prepare a good liquid for causing the electrodeposition.

Examples of the test element of the present invention include various electrodes generally used in electrochemical measurements. However, as was described in the process of electrochemically measuring a difference between the potential windows, the wider the potential window of the electrode used as the test element is, the more preferred it is, and specific examples thereof preferably include: carbon electrodes such as a glassy carbon electrode, a pyrolytic graphite electrode, a carbon paste electrode, and a carbon fiber electrode; a diamond thin film electrode; and an electron cyclotron resonance (ECR) spatter carbon electrode.

Further, an electrode having a substrate which is subjected to a pattern printing with a screen printing by using a conductive carbon ink is preferably used. The screen printing as a method of forming an electrode system is a technology for manufacturing disposable-type biosensors having uniform properties at low cost. Further, the screen printing is a convenient method for forming an electrode by using carbon which is a cheap, stable electrode material.

In addition, the form of the electrode (flat electrode, porous electrode, etc.) or the size of the electrode is not particularly limited, and can be appropriately determined depending on the kind or the amount of a measurement target substance, the amount or the properties of a measurement sample (e.g., viscosity), or the intended use or the conditions of the measurement.

### EXAMPLES

Hereinafter, the present invention is described more specifically by way of examples, and those examples are provided as references for specific embodiments of the present invention for merely describing the present invention. The illustration of those examples is conducted to describe specific embodiments of the present invention, and is not conducted to limit or restrict the scope of the present invention to be disclosed in this application. In the present invention, it should be understood that various embodiments based on the concept of this specification can be performed.

Further, all examples, except for those described in detail separately, were carried out or are able to be carried out by using a standard technology.

(Relationship Between Deposition of Chloroauric Acid and Hydrogen Overvoltage)

In the experiment of the present invention, using aqueous solutions of chloroauric acid with various concentrations, the concentrations of gold contained in the measurement solutions were each detected by (1) a method of electrochemically measuring a reduction current of gold to be deposited on an electrode and by (2) a method of electrochemically measuring a hydrogen overvoltage of gold deposited on an electrode, and respective detection limits were compared with each other.

(1) Method of Electrochemically Measuring Reduction Current of Gold to be Deposited on Electrode (Conventional Example)

Sodium tetrachloroaurate (II) dihydrate (manufactured by ALDRICH, 298174-1G) was dissolved in 1 M hydrochloric acid, whereby chloroauric acid solutions with various concentrations were prepared. Next, 40 μl of the chloroauric acid solutions with various concentrations were each added on a screen printing electrode (manufactured by Bio Device Technology Co., Ltd., DEP-EP-P) and a DPV measurement (sweep range=1.25 to 0 V, sweep rate=25 mV/s) was performed, whereby the reduction current of deposited gold was determined.

(2) Method of Electrochemically Measuring Hydrogen Overvoltage of Gold Deposited on Electrode

40 μl of the chloroauric acid solutions with various concentrations were each added on a screen printing electrode, a voltage of −0.4 V was applied to the resultant for 60 seconds, and immediately after that, a CV measurement (sweep range=−0.4 to −1.2 V, sweep rate=100 mV/s) was performed, whereby a hydrogen overvoltage value depending on the deposition amount of gold was determined.

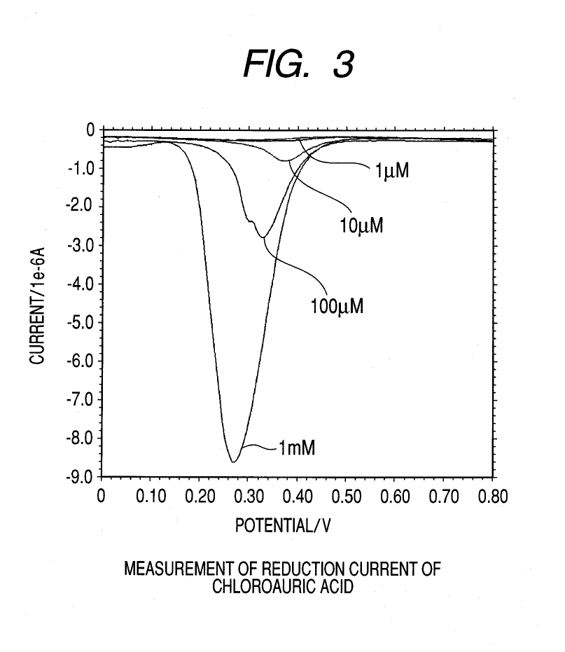

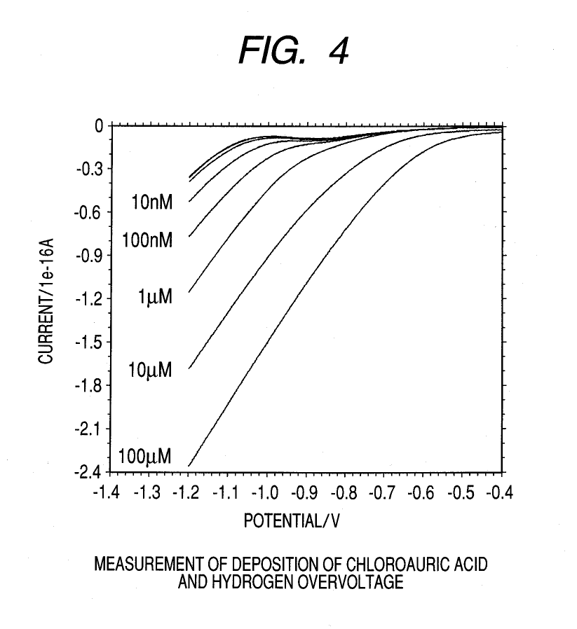

The results thereof are shown in FIGS. 3 and 4. In the case where chloroauric acid contained in the solution was electrochemically detected from the reduction current of gold to be deposited on the electrode, the detection limit thereof was 1 μM (FIG. 3). On the other hand, in the case where chloroauric acid contained in the solution was detected from the hydrogen overvoltage value of gold deposited on the electrode, the detection limit thereof was 10 nM (FIG. 4). From those results, it was found that, when chloroauric acid contained in the solution was detected, gold were able to be detected with higher sensitivity by measuring the hydrogen overvoltage which is derived from gold deposited on the electrode, rather than by measuring the electrochemical reduction reaction of gold itself.

(Relationship Between Labeling Substance to be Deposited and Hydrogen Overvoltage)

In the experiment of the present invention, using chloroauric acid solutions and chloroplatinic acid solutions with various concentrations, concentrations of gold and platinum contained in the measurement solutions were each measured by a method of electrochemically measuring a change in the hydrogen overvoltages, which was caused when gold or platinum was deposited on the electrode, and respective detection limits were compared with each other.

(1) Method of Electrochemically Measuring Hydrogen Overvoltage of Gold and Platinum Deposited on Electrode

Sodium tetrachloroaurate (II) dihydrate (manufactured by ALDRICH, 298174-1G) was dissolved in 1 M hydrochloric acid, whereby chloroauric acid solutions with various concentrations were prepared. Further, hexachloroplatinate (IV) hexahydrate (manufactured by Kishida Chemical Co., Ltd., 000-62771) was dissolved in 1 M hydrochloric acid, whereby chloroplatinic acid solutions with various concentrations were prepared.

40 μl of the chloroauric acid solutions and the chloroplatinic acid solutions with various concentrations were each added on a screen printing electrode, a voltage of −0.4 V was applied to the resultant for 300 seconds, and immediately after that, a CV measurement (sweep range=0 to −1.2 V, sweep rate=100 mV/s) was performed, whereby a change in the hydrogen overvoltage depending on the deposition amount of each metal as a change in the current at a constant voltage value was determined.

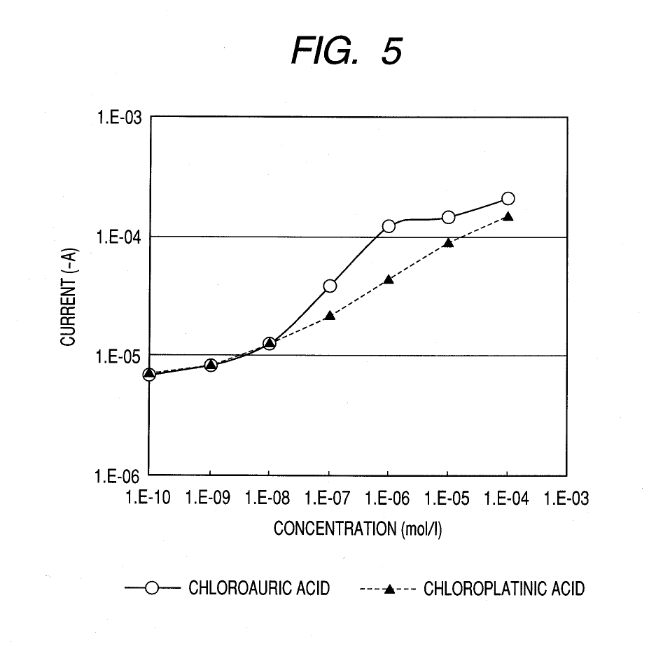

FIG. 5 illustrates calibration curves obtained in the experiment of the section (1). The current values (−1.1 V, constant) obtained from the respective CV measurements were plotted. As the concentrations of the chloroauric acid solution and the chloroplatinic acid solution become higher, the obtained current values become larger. Those results show that the deposition amount of the labeling substance deposited on the electrode can be assayed as the change in the hydrogen overvoltage. Further, it is generally known that the catalytic capacity of platinum is higher than the catalytic capacity of gold, and thus, it can be considered that the reason for the gradient of the calibration curve of chloroplatinic acid in FIG. 5 being steeper attributes to the above fact.

### Example 1

### Detection of HCG when Insoluble Magnetic Carrier Particle is Used as Support

In this example, an insoluble magnetic carrier particle was used as a support and a gold colloid was used as a labeling substance, and the detection of human chorionic gonadotropin (HCG) was performed.

(1) Immobilization of Primary Antibody on Insoluble Magnetic Carrier Particle

The immobilization of an HCG antibody (manufactured by Medix Biochemica, 5008) on an insoluble magnetic carrier particle (manufactured by VERITAS Corporation, Dynabeads M-280 Tosylactivated) was basically performed in accordance with the protocol released by VERITAS Corporation. First, 500 μl of the magnetic carrier particles were fractionated and loaded into a 2-ml Eppentube. Next, the magnetic carrier particles were precipitated by B/F separation using a magnet to remove the supernatant of the resultant, and the resultant was substituted with 500 μl of a boric acid buffer (pH 9.5). After the supernatant of the resultant was removed by B/F separation again, 1 ml of a 300 μg/ml HCG antibody was added thereto and the mixture was reacted at 37° C. for 24 hours. Subsequently, after the supernatant was removed by B/F separation, 1 ml of a 1 mg/ml BSA solution was added thereto, followed by stirring for 5 minutes. Finally, after the supernatant was removed by B/F separation, 1 ml of a 0.2 M Tris buffer containing 0.1% BSA (pH 8.5) was added thereto, and the mixture was reacted at 37° C. for 4 hours.

(2) Labeling of Secondary Antibody with Gold Colloid

9 ml of a gold colloid solution (manufactured by BBI, particle diameter of 40 nm) and 1 ml of a 20 mM boric acid buffer (pH 9) were loaded into a 50-ml test tube for centrifugation, and the mixture was gently stirred. Next, 1 ml of an α-subunit antibody (manufactured by Medix Biochemica, 6601) of 80 μg/ml human follicle stimulating hormone (FSH) (FSH α-subunit being immunologically identical to HCG α-subunit) was added to the resultant, and the mixture was left standing still at room temperature for 10 minutes. After that, 0.55 ml of 1% PEG 20000 and 1.1 ml of 10% BSA were added to the resultant, and the mixture was gently stirred. Further, centrifugation was performed at 8,000×g for 15 minutes, thereby precipitating the gold colloid and removing the supernatant. To the resultant, 20 ml of a gold colloid retention buffer (pH 8.2) (aqueous solution in which the following were dissolved in 500 ml of pure water: 5 g of BSA; 0.5 g of sodium azide; 0.25 g of PEG 20000; 1.211 g of tris(hydroxymethyl)aminomethane; and 4.383 g of sodium chloride) were added, and centrifugation was performed again. After that, the supernatant was removed, and the resultant was diluted with the gold colloid retention buffer in such a manner that the obtained human FSH α-subunit antibody labeled with a gold colloid had an absorbance (520 nm) of 6.0.

(3) Detection of HCG

180 μl of each of HCG solutions prepared to have respective concentrations and 5 μl of HCG antibody-immobilized magnetic carrier particles were loaded into a 2-ml Eppentube, and the mixture was reacted for 20 minutes while stirring. Next, after unreacted HCG was removed by B/F separation, 100 μl of the human FSH α-subunit antibody labeled with a gold colloid which was prepared to have an absorbance (520 nm) of 0.1 were added to the resultant, and the mixture was reacted for 15 minutes while stirring. After that, the supernatant of the resultant was removed by B/F separation, 40 μl of aqua regia diluted to 20% were added to the resultant, and the mixture was reacted for 3 minutes while stirring. Then, 35 μl of the obtained chloroauric acid solution were added on a screen printing electrode, and after a voltage of −0.4 V was applied to the resultant for 90 seconds, a CV measurement (sweep range=0 to −1.2 V, sweep rate=100 mV/s) was performed, whereby a change in hydrogen overvoltage was determined.

### Example 2

### Detection of HCG when Test Element is Used as Support

In this example, a screen printing electrode which was a test element was used as a support and a gold colloid was used as a labeling substance, and the detection of HCG was performed.

(1) Immobilization of Primary Antibody on Screen Printing Electrode

5 μl of a 10 μg/ml HCG antibody (manufactured by Medix Biochemica, 5008) were added on a working electrode of the screen printing electrode, and the resultant was reacted overnight at 4° C. After that, the resultant was washed with 1×PBST, whereby an HCG antibody-immobilized screen printing electrode was produced.

(2) Detection of HCG

100 μl of each of HCG solutions prepared to have respective concentrations were loaded into a 1.5-ml Eppentube, and the HCG antibody-immobilized screen printing electrode was immersed therein and was reacted therewith at ordinary temperature for 2 hours. On the other hand, 100 μl of the human FSH α-subunit antibody labeled with a gold colloid which was prepared to have an absorbance (520 nm) of 0.1 were loaded into a 1.5-ml Eppentube, and the screen printing electrode reacted with HCG was immersed therein and was reacted therewith at ordinary temperature for 2 hours. Next, the surface of the electrode was washed with 1×PBST, then 20 μl of 1 M of hydrochloric acid were added on the electrode, and after a voltage of −0.4 V was applied to the resultant for 90 seconds, a CV measurement (sweep range=0 to −1.2 V, sweep rate=100 mV/s) was performed, whereby a hydrogen overvoltage was determined.

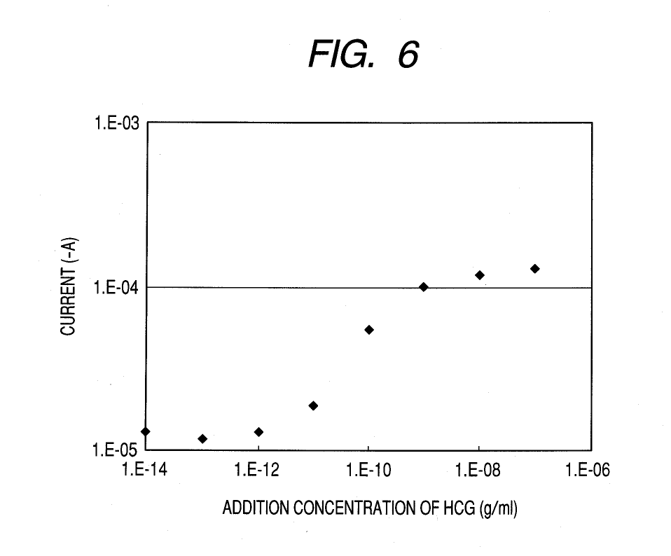

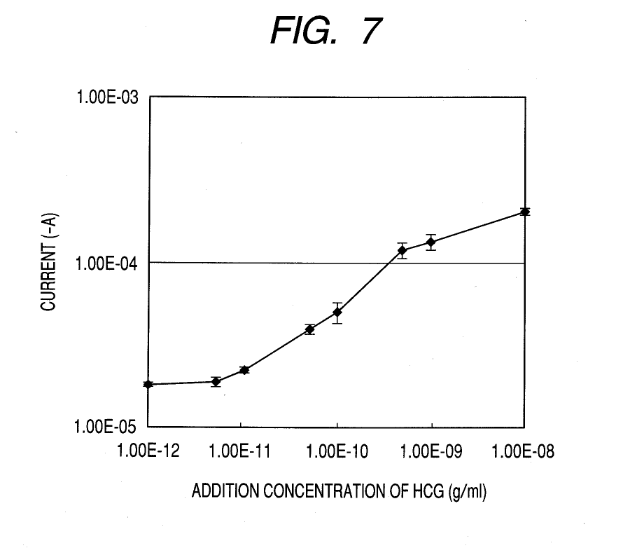

FIGS. 6 and 7 illustrate calibration curves for Examples 1 and 2, respectively, the calibration curves each showing the relationship between the addition concentration of HCG and the reduction current at an applied voltage of −1.2 V. As the concentration of HCG contained in the sample liquid becomes higher, the current value to be obtained becomes larger. Further, in both experiment results, the detection of HCG is possible from 10 pg/ml, which is highly sensitive. Thus, the amount of the test substance can be quantified by electrochemically measuring the catalyst reaction amount of gold deposited on the electrode.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2008-108155, filed Apr. 17, 2008, which is hereby incorporated by reference herein in its entirety.

1. An immunoassay for detecting or measuring a test substance contained in a sample liquid, comprising:
reacting the test substance with a first specifically binding substance which is immobilized on a support and is capable of binding specifically to the test substance;
reacting the test substance with a second specifically binding substance which is labeled with a labeling substance and is capable of binding specifically to the test substance;
eluting the labeling substance retained on the support;
depositing the eluted labeling substance on an electrode; and
measuring electrochemically a catalytic capacity of the labeling substance deposited on the electrode,
wherein the labeling substance catalyzes an electrochemical reaction of a solution.

2. The immunoassay according to claim 1, wherein:
the labeling substance comprises a conductive material having a potential window narrower than a potential window of the electrode; and
the measuring electrochemically the catalytic capacity of the labeling substance deposited on the electrode comprises measuring electrochemically a difference between a potential window of the electrode and a potential window of the electrode on which the labeling substance is deposited.

3. The immunoassay according to claim 1, wherein the labeling substance comprises a gold colloid.

4. The immunoassay according to claim 1, wherein the measuring electrochemically the catalytic capacity of the labeling substance deposited on the electrode comprises measuring oxygen overvoltage or hydrogen overvoltage.
